(19) United States
(12) Patent Application Publication
(10) Pub. No.: US 2022/0287580 A1
(43) Pub. Date: Sep. 15, 2022

(54) Wireless Hemodynamic Sensors and Methods of Using Same

(71) Applicant: Georgia Tech Research Corporation, Atlanta, GA (US)

(72) Inventors: Woon-Hong Yeo, Atlanta, GA (US); Robert Herbert, Atlanta, GA (US)

(21) Appl. No.: 17/664,167

(22) Filed: May 19, 2022

Related U.S. Application Data
(63) Continuation of application No. 17/490,813, filed on Sep. 30, 2021, now abandoned.
(60) Provisional application No. 63/085,652, filed on Sep. 30, 2020.

(51) Int. Cl. A61B 5/0215; A61B 5/00; A61B 5/024; A61B 5/026

(52) U.S. Cl. CPC A61B 5/02158; A61B 5/6862; A61B 5/02444; A61B 5/026; A61B 5/0031; A61B 2562/0247

(57) ABSTRACT

A wireless hemodynamic sensor system is provided comprising a stent and a sensor member. The stent can have an outer perimeter defining an interior volume. The sensor member can be positioned along the outer perimeter. The sensor member can comprise a first sensor positioned proximate a first end of the stent and a second sensor positioned proximate a second end of the stent. The sensor system can be configured to simultaneously measure one or more of blood pressure, pulse rate, and blood flow rate of blood passing through the interior volume.

### CROSS-REFERENCE TO RELATED APPLICATIONS

This application is a continuation of U.S. Non-Provisional application Ser. No. 17/490,813, filed on 30 Sep. 2021, which claims the benefit of U.S. Provisional Application Ser. No. 63/085,652, filed on 30 Sep. 2020, which are incorporated herein by reference in their entireties as if fully set forth below.

### FIELD OF THE DISCLOSURE

The various embodiments of the present disclosure relate generally to sensors, and more particularly to sensors for monitoring hemodynamic properties in a user.

### BACKGROUND

Vascular diseases are the leading cause of death, accounting for over 30% of deaths worldwide. Diseases and conditions, such as hypertension, atherosclerosis, and aneurysms, occur throughout the vascular system, including in arteries from a few millimeters to centimeters in diameter with varying curvature. Blood pressures and flow rates, among other hemodynamics, are monitored to follow disease progression and treatment. However, current hemodynamic monitoring methods, including angiography, magnetic resonance imaging, Doppler ultrasound, and catheterization, provide narrow and incomplete views of vascular health due to limited and repetitive monitoring periods and patient immobilization. Although continuous hemodynamic monitoring has been shown to improve patient outcomes, existing clinical devices offer limited sensing capabilities due to their bulky packages and rigid materials. These devices are suitable for only pressure monitoring within the heart, abdominal aneurysms, and pulmonary artery, and are incompatible with other arteries. Overall, the development of vascular electronics for arterial sensing has been limited by strict requirements for implantation and operation, including offering sufficient wireless capabilities with a flexible, miniaturized, and low-profile system that affixes itself within an artery and is compatible with minimally invasive catheter implantation. Advances in stretchable and flexible electronics offer a means of forming wireless arterial sensors. One recent work targeted vessel anastomosis and demonstrated a cuff-type, flexible pulse sensor that is sutured outside of an artery with a wireless antenna extending outwards. For catheter compatibility, works have developed stent-based systems since stents provide an implantable backbone and are commonly used, with over 3 million implanted in cardiovascular arteries each year. Stent-based systems have attached wireless sensors to stents and have used stents as wireless antennas. However, all existing devices have shortcomings in requiring memory modules, displaying low wireless distances, or showing fragility during implantation.

### BRIEF SUMMARY

In accordance with one aspect of the present disclosure, a wireless hemodynamic sensor system is provided comprising a stent and a sensor member. The stent can have an outer perimeter defining an interior volume. The sensor member can be positioned along the outer perimeter. The sensor member can comprise a first sensor positioned proximate a first end of the stent and a second sensor positioned proximate a second end of the stent. The sensor system can be configured to simultaneously measure one or more of blood pressure, pulse rate, and blood flow rate of blood passing through the interior volume.

In any of the embodiments disclosed herein, the outer perimeter of the stent can comprise a plurality of conductive loops. Each of the plurality of conductive loops can be coupled to an adjacent conductive loop via a non-conductive connector.

In any of the embodiments disclosed herein, the outer perimeter can form an inductive antenna.

In any of the embodiments disclosed herein, the sensor member can comprise a first electrode, a second electrode, and a dielectric layer positioned between the first and second electrodes.

In any of the embodiments disclosed herein, the sensor member can be electrically coupled to the stent via a first connection to the first electrode proximate the first end of the stent, a second connection to the first electrode proximate the second end of the stent, and a third connection to the second electrode proximate a location between the first and second sensors.

In any of the embodiments disclosed herein, each of the first, second, and third connections can be insulated with PDMS.

In any of the embodiments disclosed herein, the first sensor can be configured to operate within a first resonant frequency range, the second sensor can be configured to operate within a second resonant frequency range, such that the first resonant frequency range does not overlap with the second resonant frequency range.

In any of the embodiments disclosed herein, the system can be configured to measure a pressure gradient between the first sensor and the second sensor.

In any of the embodiments disclosed herein, the blood pressure, pulse rate, and blood flow rate measurements may not be degraded if the sensor member are bent with a radius of curvature of 1.5 mm.

In any of the embodiments disclosed herein, the first and second sensors can be capacitive pressure sensors.

In any of the embodiments disclosed herein, the plurality of conductive loops can comprise stainless steel.

In any of the embodiments disclosed herein, the plurality of conductive loops can be coated in gold.

In any of the embodiments disclosed herein, the nonconductive connectors can comprise polyimide.

In any of the embodiments disclosed herein, each of the plurality of conductive loops can have an S-shape to facilitate stretching of the stent.

In accordance with another aspect of the present disclosure, a wireless hemodynamic sensor system is provided comprising a stent and a sensor member. The stent can have an outer wall defining an interior volume. The stent can be configured to be placed in a blood vessel of a patient. The sensor member can be positioned along inner surface of the outer wall. The sensor member can comprise a first capacitive pressure sensor positioned proximate a first end of the stent and a second capacitive pressure sensor positioned proximate a second end of the stent. The first and second sensors can be configured to measure blood pressure, blood flow rate, and pulse rate of blood flowing through the blood vessel.

In any of the embodiments disclosed herein, the outer wall of the stent can comprise a plurality of conductive loops. Each of the conductive loops can be coupled to an adjacent conductive loop via a nonconductive connector. The outer wall can form an inductive antenna capable of being interrogated by a second external inductive antenna.

In any of the embodiments disclosed herein, the sensor member can comprise a first electrode electrically coupled to the first and second ends of the stent, a second electrode electrically coupled the stent between the first and second ends of the stent, and a dielectric material between the first and second electrodes.

In any of the embodiments disclosed herein, the sensor system can be capable of simultaneously measuring one or more of blood pressure, blood flow rate, and pulse rate of blood flowing through the blood vessel if the sensor member is bent at a radius of 1.5 mm.

These and other aspects of the present disclosure are described in the Detailed Description below and the accompanying drawings. Other aspects and features of embodiments will become apparent to those of ordinary skill in the art upon reviewing the following description of specific, exemplary embodiments in concert with the drawings. While features of the present disclosure may be discussed relative to certain embodiments and figures, all embodiments of the present disclosure can include one or more of the features discussed herein. Further, while one or more embodiments may be discussed as having certain advantageous features, one or more of such features may also be used with the various embodiments discussed herein. In similar fashion, while exemplary embodiments may be discussed below as device, system, or method embodiments, it is to be understood that such exemplary embodiments can be implemented in various devices, systems, and methods of the present disclosure.

### BRIEF DESCRIPTION OF THE DRAWINGS

The following detailed description of specific embodiments of the disclosure will be better understood when read in conjunction with the appended drawings. For the purpose of illustrating the disclosure, specific embodiments are shown in the drawings. It should be understood, however, that the disclosure is not limited to the precise arrangements and instrumentalities of the embodiments shown in the drawings.

FIGS. 1A-G provide an overview of a fully implantable, wireless vascular electronic system with printed sensors for wireless monitoring of hemodynamics, in accordance with an exemplary embodiment of the present disclosure. FIG. 1A provides an illustration of the exemplary implantable electronic components. FIG. 1B provides an illustration of an exemplary inductive stent design using conductive Au loops and nonconductive PI connectors to achieve a current path resembling a solenoid (left) and an SEM image of the stent (right). FIG. 1C illustrates layers of the exemplary soft pressure sensor using a printed dielectric layer (left) and photo of index finger holding a simultaneous flow and pressure sensor (right). FIG. 1D provides an illustration of minimally invasive catheter deployment and balloon expansion of the exemplary wireless vascular stent. FIG. 1E provides an illustration of initial and expanded state of the exemplary sensor-integrated stent system. FIG. 1F provides an illustration of the exemplary wireless stent system implanted in the right iliac artery of living rabbit. FIG. 1G provides an illustration of the exemplary wireless design and sensing scheme to simultaneously monitor pressure, pulse rate, and flow.

FIGS. 2A-L illustrate the design, fabrication, and characterization of wireless stent, in accordance with an exemplary embodiment of the present disclosure. FIG. 2A provides an illustration of fabrication steps for an exemplary multi-material, inductive stent. FIG. 2B illustrates the layout of an exemplary stent design using conductive Au loops and nonconductive PI connectors. FIG. 2C provides SEM images of an exemplary stent structure showing PI connectors (left) and enlarged views of the PI connector showing separation of Au loops (right). FIG. 2D provides a cross-sectional image of an exemplary stent strut showing the layers of SS, Au, and parylene. FIG. 2E illustrates balloon expansion of the exemplary wireless stent; resistance shows a minimal increase while inductance increases; photos show the progression of expansion on the balloon. FIG. 2F provides a plot of increased stent diameter according to balloon pressure. FIG. 2G illustrates magnitude of S₁₁ parameter at the resonant frequency at different distances for the exemplary wireless stent and a Cu coil, in which a single-loop, unmatched antenna was used for wireless reading, and in which magnitude decreases with distance and becomes unreadable below the noise level. FIG. 2H provides a chart showing maximum wireless readout distance achieved by each exemplary stent with a single-loop antenna, in which matching the antenna enhances readout distance. FIG. 1I provides a plot of wireless frequency sweep of S₁₁ parameter from an exemplary stent and sensor, in which larger distances result in less pronounced resonant dips. FIGS. 2J-L provides plots of measurement of axial stiffness (FIG. 2J), bending stiffness (FIG. 2K), and radial stiffness (FIG. 2L) of the exemplary wireless stent, in which a comparison is included with a commercial stent, a wireless stent without PI connectors, and an inductive stent design with SS connectors.

FIGS. 3A-L illustrate the fabrication and characterization of exemplary soft pressure sensors, in accordance with an exemplary embodiment of the present disclosure. FIG. 3A provides an exploded view of sensor layers following sequential aerosol jet printing of PI, AgNP, and PDMS inks, in which the top and bottom electrodes are printed separately and laminated together. FIG. 3B provides a photograph of aerosol jet nozzle for printing sensors. FIG. 3C provides an exemplary soft pressure sensor with interconnects held on a fingertip. FIG. 3D provides an SEM image of the bottom electrode of an exemplary sensor comprising a support PI layer, conductive AgNP layer, and dielectric PDMS layer. FIG. 3E provides profile measurement of the interconnect (left), electrode (center), and enlarged view of the dielectric layer (right). FIG. 3F illustrates pressure sensitivity is enhanced by using a dielectric layer of patterned PDMS lines compared to a solid thin film with similar thicknesses. FIG. 3G illustrates sensor capacitance compares well with pressure waves over time (left) and sudden, large pressure changes (right), wherein the sensor shows an immediate response to a 300 mmHg pressure increase and decrease. FIG. 3H illustrates pressure cycling from 0 to 1000 mmHg for 2,500 cycles showed minimal change in sensor performance. FIG. 3I illustrates sensor response during balloon expansion with the exemplary wireless stent. FIG. 3J provides a demonstration of sensor twisting and bending without failure. FIG. 3K illustrates sensor response to pressure when in a state of bending, in which sensitivity stays constant at a 1.5 mm bending radius and maintains sensing capabilities beyond a 0.25 mm bending radius. FIG. 3L provides a comparison of the sensor to prior works on pressure sensing during bending, in which the exemplary sensor of the present disclosure demonstrates pressure sensing at the lowest bending radius among capacitive pressure sensors and second-lowest among both sensor types.

FIGS. 4A-M provide a demonstration of wireless sensing of pressure pulse, and flow, in accordance with an exemplary embodiment of the present disclosure. FIG. 4A provides a photograph of an exemplary wireless sensing system advancing through a guide catheter. FIG. 4B provides an exemplary expanded stent and sensor in artery model, in which the inset shows a cross-section of the low-profile electronics, and enlarged views show the expanded stent structure and PI connectors. FIG. 4C provides a schematic of wired and wireless sensing methods in artery model. FIG. 4D illustrates sensor capacitance during pulsatile flow in artery model with an enlarged view of pressure waveform. FIG. 4E provides a summary of wired sensor response during various flow rate levels, in which capacitance increases linearly with pressure, indicating flow has a minimal effect. FIG. 4F provides a plot of wireless resonant frequency sweeps at different pressures, wherein the resonant frequency decreases with increasing pressure. FIG. 4G provides wireless pressure sensing in artery model with an enlarged view of the pulsatile wave. FIG. 4H provides a summary of wireless pressure sensing of average, maximum, and minimum pressures during pulsatile flow. FIG. 4I provides pulse rate detection during two flow conditions, wherein the wireless sensor detects a similar pulse rate to a commercial pressure sensor. FIG. 4J provides a wireless stent integrated with dual pressure sensor for monitoring of flow, in which the two sensors provide monitoring of two resonant frequencies, enabling real-time pressure gradient (ΔP) monitoring. FIG. 4K provides a summary of wireless flow monitoring comparing the pressure gradients monitored by the exemplary wireless sensor of the present disclosure and commercial sensors. FIG. 4L provides a plot of a wireless signal from the exemplary device when operated in air and saline, in which the conductive surrounding lowers the wireless signal quality. FIG. 4M provides a plot of wireless readout distances in air, saline, and saline plus tissue.

FIGS. 5A-H illustrate the results of an in vivo study of implantation of a wireless sensing device, in accordance with an exemplary embodiment of the present disclosure. FIG. 5A photographs of an exemplary 1.5 mm diameter stent and sensor advanced through the guide catheter and expanded on a balloon. FIG. 5B provides photographs of an exemplary expanded stent and sensor. FIG. 5C provides a schematic of in vivo catheter implantation in a rabbit where the wireless device is guided on a balloon catheter from the common carotid artery to the right iliac artery. FIG. 5D provides fluoroscopy images showing the target site in the right iliac artery, in which the exemplary stent and sensor are guided to the right iliac artery followed by expansion and removal of the catheter. FIG. 5E provides images of an exemplary stent and sensor implanted in the right iliac artery. FIG. 5F provides a plot of wireless frequency sweeps of the exemplary implanted sensor before implantation and after removal, in which signals show a minor change three months later. FIG. 5G provides a plot showing resonant frequency before and after expansion matches theoretical prediction. FIG. 5H provides a plot of pressure detection when the harvested device is wirelessly interrogated in an artery model.

FIGS. 6A-E provide illustrations of conventional stent patterns and exemplary inductive stent patterns of the present disclosure. FIG. 6A illustrates conventional stent pattern including loops and connectors of a single material. FIG. 6B illustrates that inductive stent pattern can require the removal of connectors for higher inductance. FIG. 6C illustrates a multi-material inductive stent using steel loops and PI connectors, in accordance with some exemplary embodiments of the present disclosure. FIG. 6D provides an enlarged view of an exemplary PI connector. FIG. 6E illustrates the design of exemplary loops.

FIGS. 7A-E illustrate variations in inductive stent loop design, in accordance with various exemplary embodiments of the present disclosure. FIG. 7A illustrates an initially tested pattern using a serpentine loop to form a solenoid-like structure. FIG. 7B illustrates a 0° pattern using individual columns of loops that readily expanded. FIGS. 7C-E illustrate 15°, 20°, and 30° patterns offering a higher density of turns while maintaining expansion capabilities, in which a larger angle offers more turns per length while the expansion diameter decreases.

FIGS. 8A-C illustrate a comparison of multiple stent versions, in accordance with various exemplary embodiments of the present disclosure. FIG. 8A illustrates a large stent with single pressure sensor. FIG. 8B illustrates a large stent with two pressure sensors for flow sensing. FIG. 8C illustrates a small stent with single pressure sensor.

FIGS. 9A-C illustrate the effect of bending on an exemplary wireless stent. FIG. 9A provides a plot of the magnitude of S11 change at resonance for a stent during bending of 0°, 20°, 40°, and 60°, in which as the degree of bending increases, the signal decreases due to deformation of the stent. FIG. 9B provides a plot of the magnitude of S11 change at resonance at increasing radial distances for a stent in a straight position (0° bending) and a stent in 30° bending, in which the two stents demonstrated similar signals. FIG. 9C provides the readout distance of stents in a straight position (0° bending) and a stent in 30° bending, in which radial distance were similar despite bending, but axial distance decreased significantly when bending the stent.

FIG. 10 illustrates soft sensor fabrication, in accordance with various exemplary embodiments of the present disclosure, in which the bottom and top electrode are printed with PI and AgNP, PDMS is printed onto the bottom electrode to act as the dielectric layer, the layers are transferred and laminated together to form a parallel-plate structure, and the sensor is encapsulated in elastomer.

FIGS. 11A-D provide an illustration of the effects of bending an exemplary sensor of the present disclosure. FIG. 11A provides a photograph of a printed pressure sensor in a flat state. FIG. 11B provides a photograph of an exemplary sensor conforming to a 0.5 mm bending radius. FIG. 11C provides a plot of sensor capacitance before, during, and after bending to a 0.5 mm radius. FIG. 11D provides a photograph of sensor and interconnects when folded together by tweezers.

FIG. 12 provides an illustration of the attachment of a stent and sensor, in accordance with various exemplary embodiments of the present disclosure, in which the sensor is attached at points along the length of the stent, wherein there are three electrical connections that are insulated with PDMS, and additional attachment points are placed along the interconnects of the sensor.

### DETAILED DESCRIPTION

To facilitate an understanding of the principles and features of the present disclosure, various illustrative embodiments are explained below. The components, steps, and materials described hereinafter as making up various elements of the embodiments disclosed herein are intended to be illustrative and not restrictive. Many suitable components, steps, and materials that would perform the same or similar functions as the components, steps, and materials described herein are intended to be embraced within the scope of the disclosure. Such other components, steps, and materials not described herein can include, but are not limited to, similar components or steps that are developed after development of the embodiments disclosed herein.

At a high level, disclosed herein is a wireless stent platform integrated with soft sensors to meet implantation and operation requirements. The device can be wirelessly operated by inductive coupling to offer real-time, simultaneous monitoring of pressure, pulse rate, and flow, which offers an opportunity to detect a wide range of vascular conditions. A laser machining process to form a multi-material inductive stent is also described, which addresses a key challenge of enabling wireless connectivity while maintaining critical stent mechanics. The soft pressure sensors can be fully aerosol jet printed and conformally integrated with the stent. The use of a printed elastomer pattern as the dielectric can enable fast response times and pressure sensing even when bending at a radius of as little as 0.25 mm, which is a key advancement as flexible pressure sensors often are not demonstrated to sense during bending or degrade at bending radii as large as tens of millimeters. In an exemplary embodiment, the wireless device can be compatible with conventional stenting procedures and exhibits a 5.5 cm and 3.5 cm readout distance in air and blood, which is a 2-3 times improvement in the wireless distance over existing stent-based devices. Device performance was evaluated in a biomimetic silicone artery with pulsatile flow. Further, an in vivo study in a rabbit model demonstrates minimally invasive catheter implantation in an iliac artery with carotid access.

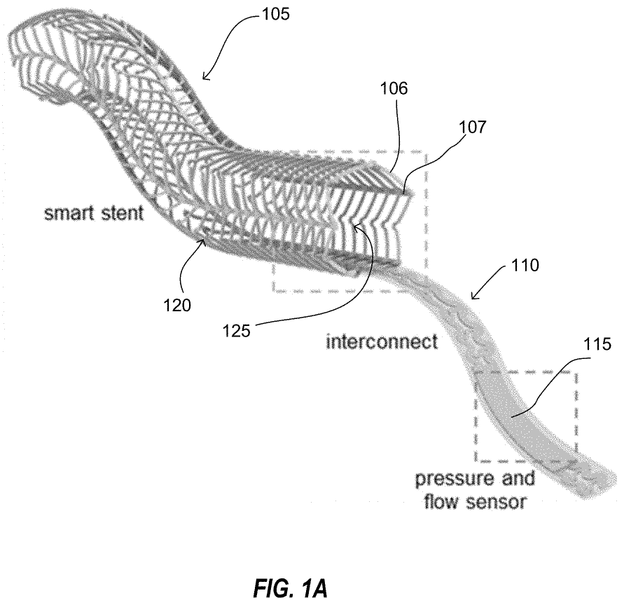

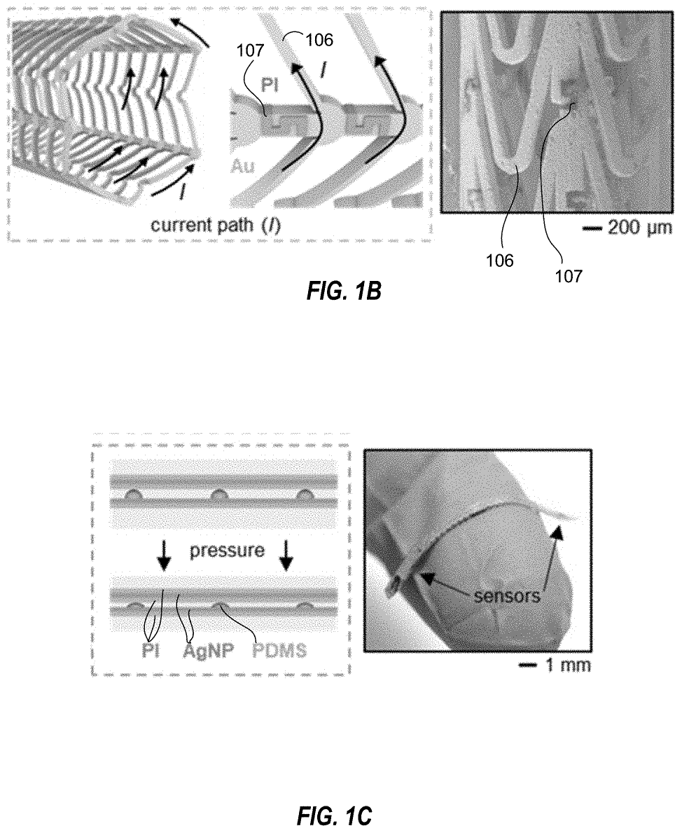

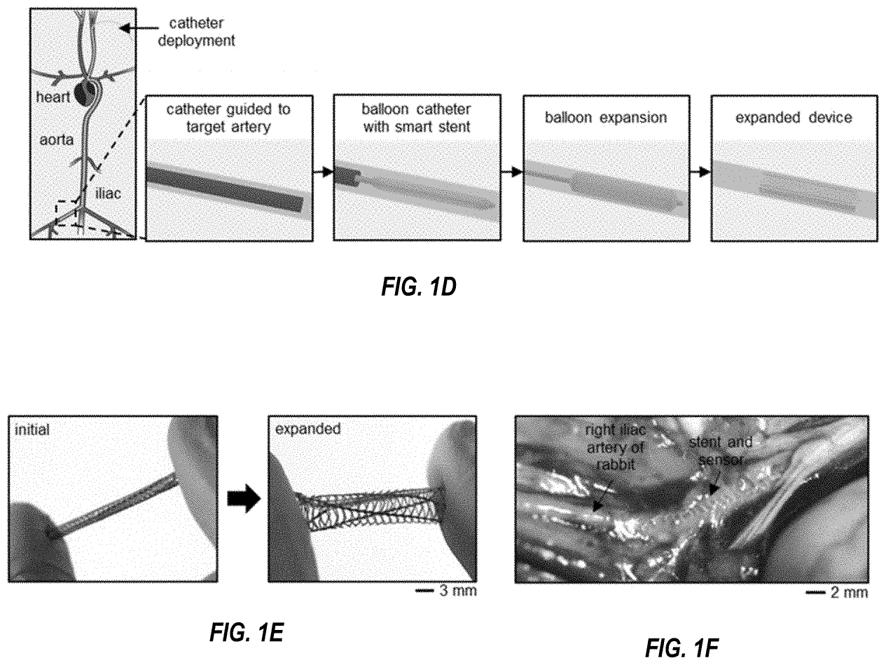

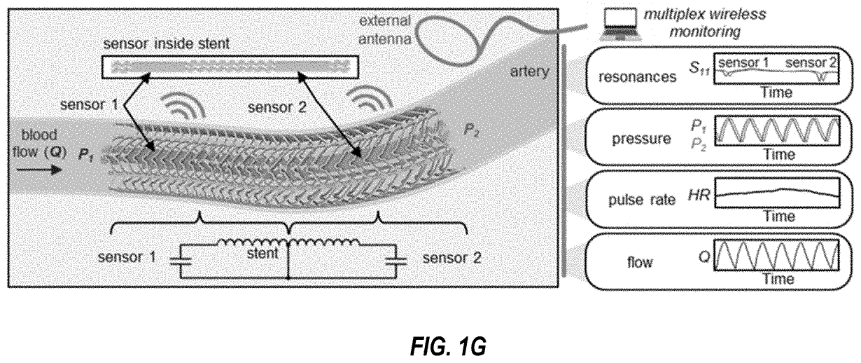

As shown in FIG. 1A, an exemplary embodiment of the present disclosure provides an implantable wireless device comprising an inductive smart stent 105 integrated with a sensing member 110 having soft, capacitive sensors 115. The stent platform can offer wireless monitoring of capacitive sensors while providing a reliable structure for implantation. The stent structure's main design and fabrication challenges can offer sufficient wireless capabilities without deviating from typical stent mechanical properties. To accomplish this, a multi-material stent 105 is employed having an outer wall/perimeter 120 defining an interior volume 125. The outer wall can be formed by a plurality of conductive loops 106 and nonconductive connectors 107 to achieve a conductive pathway resembling a solenoid and serving as an inductive antenna. An SEM image in FIG. 1B shows the fabricated stent. To unobtrusively sense hemodynamics (e.g., one or more of pulse rate, flow rate, and pressure), a sensing member 110 comprising soft, low-profile pressure sensors 115 is laminated on the inner surface of the outer wall 120 of the stent 105. The capacitive sensors 115, shown in FIG. 1C on an index finger, employ a top electrode, a bottom electrode, and a structured dielectric layer for enhanced sensitivity and response time. The wireless device can be compatible with catheter deployment, including delivery through a guide catheter and balloon expansion, as illustrated in FIG. 1D. The exemplary integrated stent and sensor shown in FIG. 1E have an initial diameter of 2 mm before expansion up to 5 mm, though the disclosure is not so limited. Similar to conventional stents, the wireless device can be readily adaptable for varying artery sizes of the user/patient. Owing to this adaptability and optimized mechanics, the device can be implanted via a minimally invasive catheter into a living rabbit's 1.93 mm diameter iliac artery (FIG. 1F). For wireless sensing, the integrated stent 105 and sensors 110 can form inductor-capacitor (LC) circuits with a resonant frequency dependent on pressure, as shown in FIG. 1G.

For the LC circuit, the stent 105 forms the inductor while the sensor 115 forms the capacitor. Here, the equation for inductance of the stent (L) is estimated as a solenoid by:

$$L = \frac{μ {N}^{2} π {d}^{2}}{4 l} \qquad ( 1 )$$

where is μ magnetic permeability, N is the number of loops, d is stent diameter, and l is stent length. Experimental results indicated this estimate to be sufficient, with the stent inductance slightly lower. Sensor capacitance (C) is given by the below equation adapted from a parallel-plate capacitor:

$$C = \frac{{ε}_{r} {ε}_{0} A}{d} \qquad ( 2 )$$

where εᵣ is relative permittivity, ε₀ is permittivity of free space, A is overlapping area of the electrode plates, and d is the separation distance. In the printed sensor, permittivity is a function of the fraction of PDMS and air. When pressure is applied, thickness of the dielectric layer of PDMS changes and causes a change in capacitance. Additionally, the overall dielectric constant slightly changes as the PDMS deforms and volume of air decreases.

The stent 110 and sensors 115 connected together form an LC circuit with a resonant frequency (f) given by:

$$f = \frac{1}{2 π \sqrt{LC}} \qquad ( 3 )$$

By monitoring resonant frequency, pressure changes are determined. For wireless reading, increasing quality factor (Q) will increase readout distance. Quality factor is given by:

$$Q = \frac{1}{R} \sqrt{\frac{L}{C}} \qquad ( 4 )$$

where R is the resistance of a circuit. By reducing resistance with a gold coating, the readout distance of the stent is improved.

While pressure monitoring can be performed with only one sensor 115, placing a pressure sensor 115 at each end shares the stent 105 and forms two LC circuits with distinct resonant frequencies. Each of these two sensors 115 can operate at non-overlapping resonant frequency ranges, i.e., non-overlapping frequency bands centered on distinct resonant frequencies. To detect both upstream and downstream pressures, two sensors can be used to monitor a pressure gradient across the length of the stent, which can allow for detecting flow rate changes. The resonant frequency of each circuit can be wirelessly monitored with the S₁₁ parameter via an external loop antenna and vector network analyzer (VNA). Overall, the wireless system can enables real-time, simultaneous monitoring of pressure, pulse rate, and flow through the blood vessel of a user.

Fabrication and Characterization of a Wireless Stent

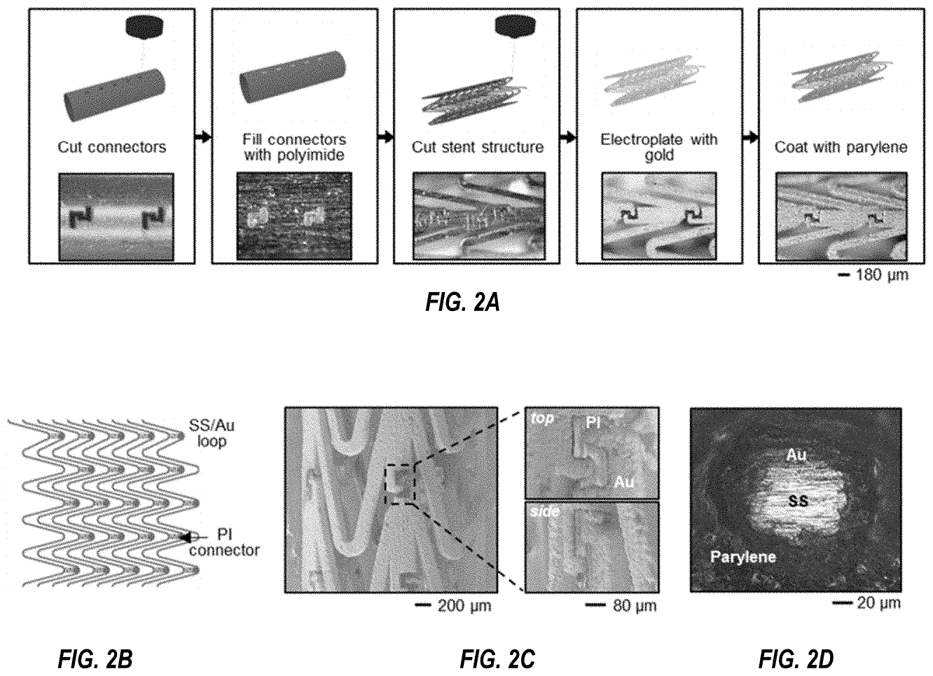

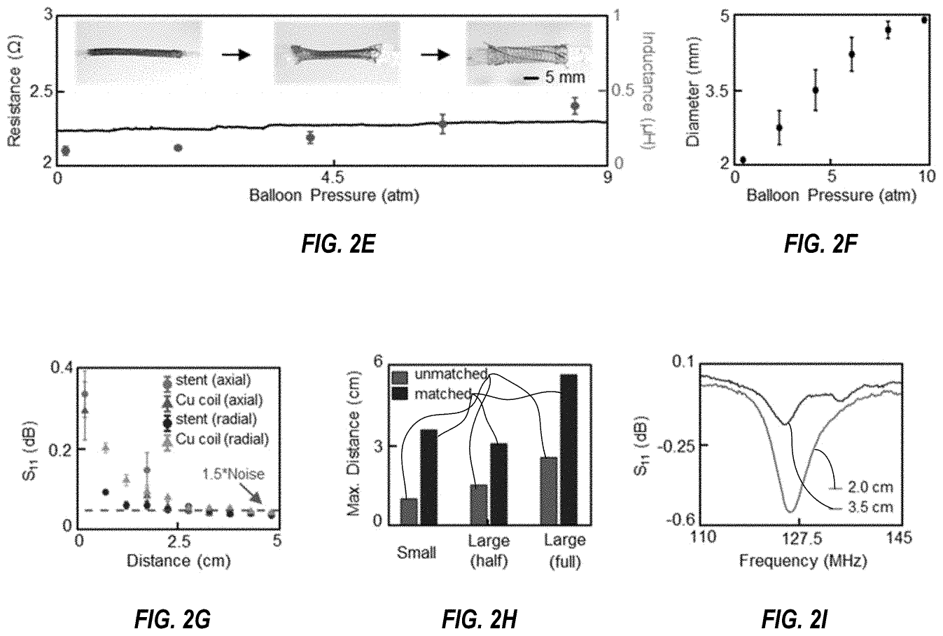

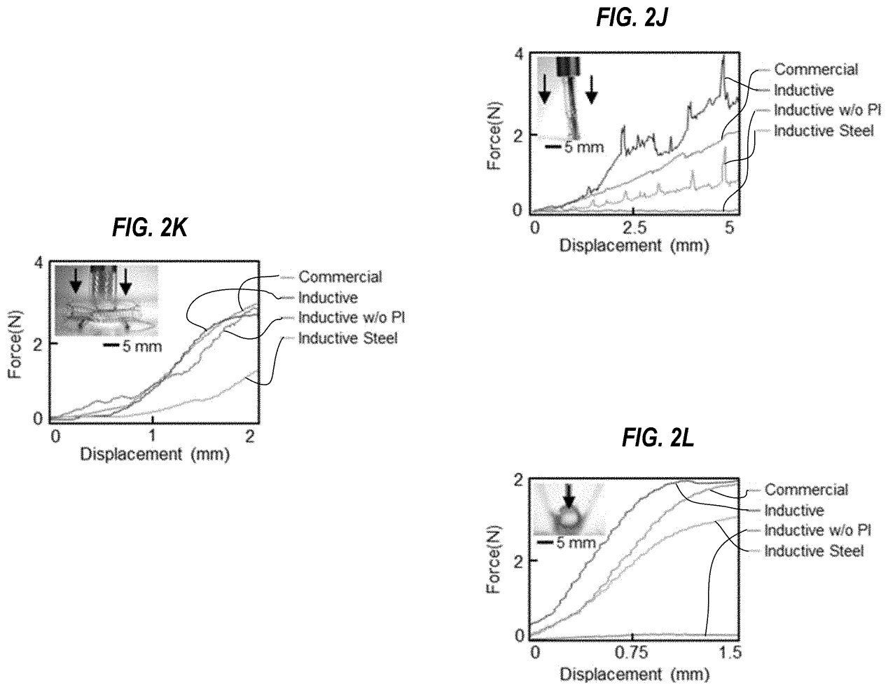

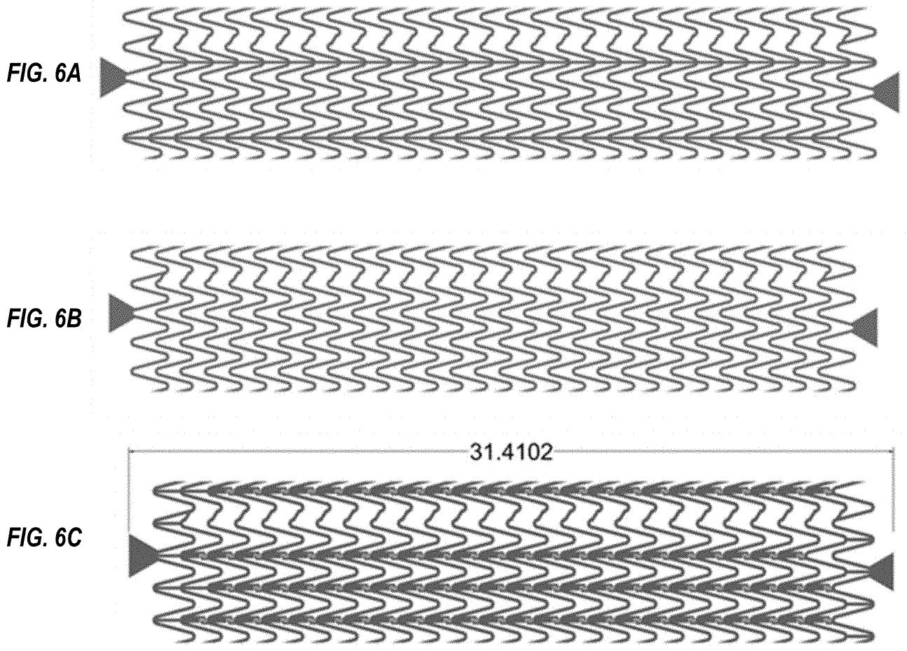

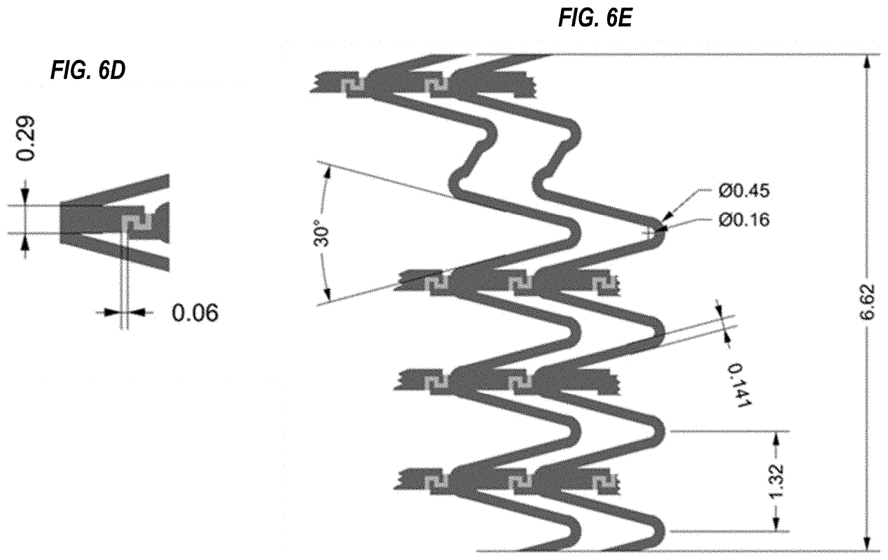

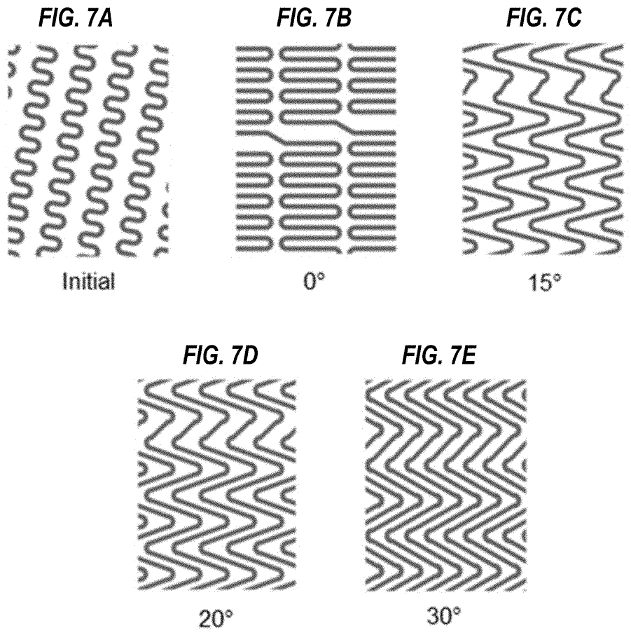

Stent design and materials were evaluated to reconfigure a conventional stent as a wireless platform. A conventional stent is formed by loops and connectors of a single material. By removing connectors and organizing loops as a continuous path, a solenoid-like design is achieved, but this design is detrimental to stent mechanics and compatibility with balloon expansion (FIGS. 6A-E). Instead, here an inductive stent design and fabrication process is developed to use conductive loops 106 and nonconductive connectors 107, which can prevent electrical shorting between adjacent loops 106. An exemplary fabrication method is shown in FIG. 2A, which relies on laser machining a cylindrical tube, which is a common stent fabrication strategy (details in Materials Methods below). First, stainless steel (SS) tubing is laser machined to remove material from the connector location before being dip-coated in polyimide (PI) to fill the connectors. After curing PI, the overall stent structure is laser machined and then electropolished to remove impurities and smooth surfaces. To enhance the quality factor of the LC circuit, the resistance of the stent can be decreased from 25Ω to 2Ω by electroplating a 25 μm thick layer of gold (Au) onto the steel surfaces. A 25 μm-thick layer of parylene, which has been previously shown to be biocompatible and hemocompatible, can be deposited to insulate the stent 105 and reinforce connectors 107. FIG. 2B illustrates an exemplary stent design using conductive loops 106 and nonconductive connectors 107 to achieve a solenoid-like structure (details of which are shown in FIGS. 6A-E). The exemplary stent discussed below uses 27 loops with a wire width of 120 μm and a length of 28 mm, though the disclosure is not so limited and can employ any number of loops with various lengths and wire widths. Indeed, the loop pattern can be varied to accommodate different diameters and inductances, as shown in FIGS. 7A-E). While a variety of connector designs may fail to endure laser machining, an “S” shape of PI with interlocking steel hooks as shown in the figures can be durable while adjacent insulating loops. Further while some connectors fail when stretched due to insufficient adhesion between PI and steel, the “S” shape reinforces the interface during stretching by the steel hooks compressing onto the center, the horizontal portion of the PI “S.” An SEM image shown in FIG. 2C shows the inductive stent 105 with enlarged views of the optimized connector 107. A 60 μm trace width of PI accommodates electroplating of the Au layer without electrically shorting across the connector 107. A cross-section image in FIG. 2D shows the multilayer coating of a stent strut without delamination. A collected set of photos and data in FIGS. 2E and 2F demonstrates balloon catheter expansion of the inductive stent from a diameter of 2 mm to 5 mm with pressure below 10 atm, which is comparable with conventional stents. The expansion can increase inductance from 0.15 to 0.46 μH, which compares well with theoretical expectations, and minimally increases resistance as the loops deform. Stent photos in FIG. 2E show the PI connectors enable uniform expansion and loop spacing. For comparison, removing the PI connectors causes distortion during expansion as a result of lowered axial stiffness. While the stent shown uses 27 loops, the density and number of loops can be increased by reducing wire thickness, increasing inductance, and lowering expansion pressure. However, the addition of loops can increase resistance due to a longer wire length, which diminishes wireless performance. A key benefit of reducing the strut thickness of the stainless steel base is to reduce the overall strut thickness of the wireless stent. Currently, the stent shows an average strut thickness of 190 μm. Future A thinner stainless steel strut and minimal coating thickness can be used because thicker stents have shown lower endothelialization and higher risk of restenosis and complications. To demonstrate adaptability and widespread application for arteries, a smaller and thinner stent with an initial diameter below 1.5 mm and an expanded diameter up to 3 mm was fabricated with a strut (115 μm in thickness) and evaluated alongside the larger stent.

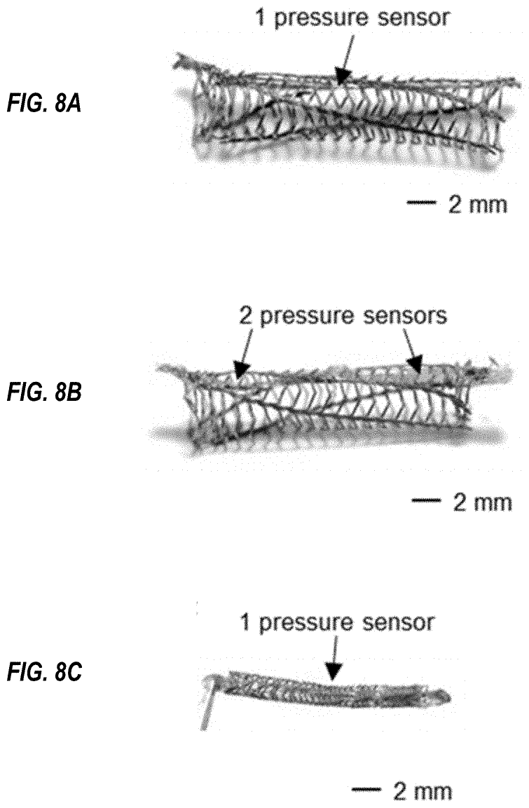

For wireless performance characterization, a loop reader antenna connected to a VNA recorded the S₁₁ parameter of the expanded stent integrated with a printed capacitive sensor. The 5 mm diameter stents with one pressure sensor and with two pressure sensors for flow sensing, as shown in FIGS. 8A-C, were tested, along with the 3 mm diameter stent with one pressure sensor. Wireless readout distance of the stent in the air was measured in radial and axial directions, and compared to an identically dimensioned solenoid inductor formed with copper (Cu) wire. FIG. 2G compares the magnitude of the S₁₁ parameter at resonance for the Cu coil and stent. The Cu coil achieved distances of 2 cm and 3 cm in the axial and radial direction, respectively, while the stent reached 1.5 cm and 2.0 cm. The distance was improved by tuning the external reader antenna with a capacitor, enabling radial distances of 5.5 cm for a large stent, 3 cm for a large stent with two sensors, and 3.5 cm for a small stent as shown in FIG. 2H. FIG. 2I shows the decrease in the signal magnitude of the frequency sweep at increasing distances. The wireless signal indicates a low power transfer efficiency due to the small stent size and an unoptimized external reader system coupled with the device. The external electronics can be improved with impedance matching methods and an optimized frequency range based upon stent dimensions, implant depth, and tissue absorption to improve device functionality and communication distance through biological tissues.

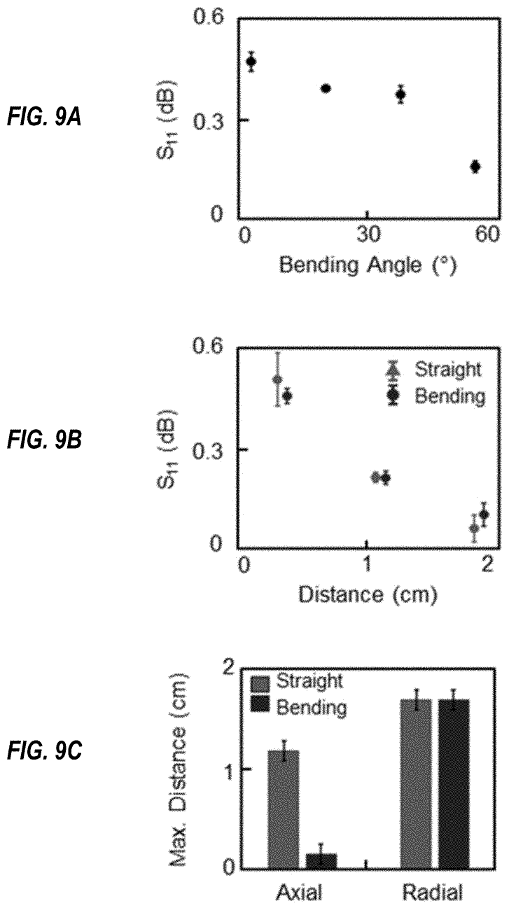

Since implant locations in arteries may require bending of the stent, wireless performance was additionally observed when the stent is subject to bending. Wireless communication distance during bending of the stent to 30° significantly affected the axial direction while the radial direction remained unchanged, as shown in FIGS. 9A-C. This occurs due to bending, causing degradation of axial alignment as axial distance increases while not affecting radial alignment. However, as the degree of bending increases, the wireless signal can also degrade in the radial direction. Along with enhancing wireless performance, the multi-material design significantly improves stent mechanical properties compared to prior works. The key implantation criteria, including axial, bending, and radial stiffness, were measured and compared among four stents: an inductive stent with PI connectors, an inductive stent without connectors, an inductive stent with only steel, and a commercial stent. As shown in FIG. 2J, the PI connectors can be useful in providing axial stiffness comparable to a commercial stent. Although the brief increases in force result from connectors buckling, the overall stiffness of the inductive stent and commercial stent is similar. Bending stiffness was substantially identical for all stents except the steel inductive stent due to the lack of Au coating, since increasing the thickness of the stent wires increases bending stiffness (FIG. 2K). FIG. 2L shows similar radial stiffness of the inductive stent and commercial stent. The axial and radial collapses of the inductive stent without connectors indicate the additional structural integrity that can be provided by the PI connectors.

Design, Fabrication, and Characterization of a Soft Pressure Sensor

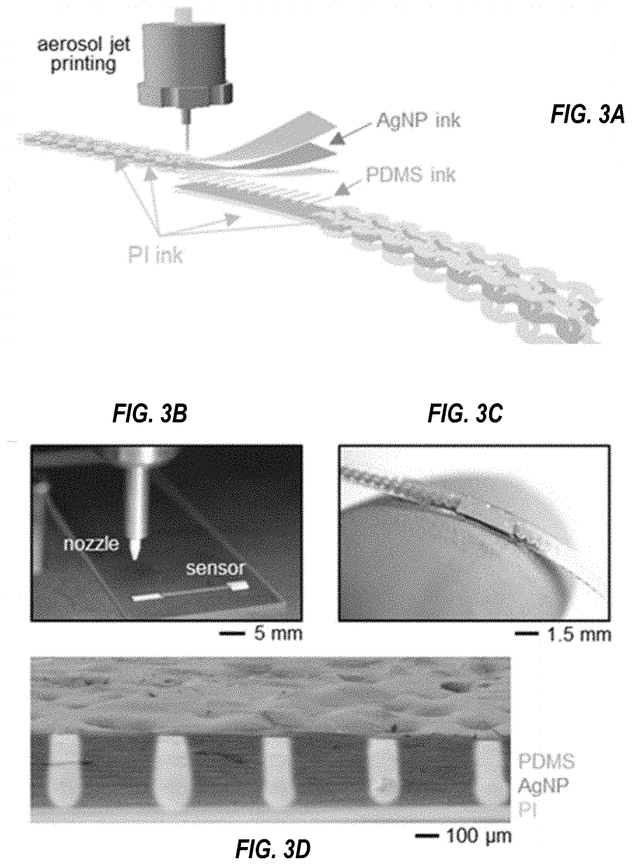

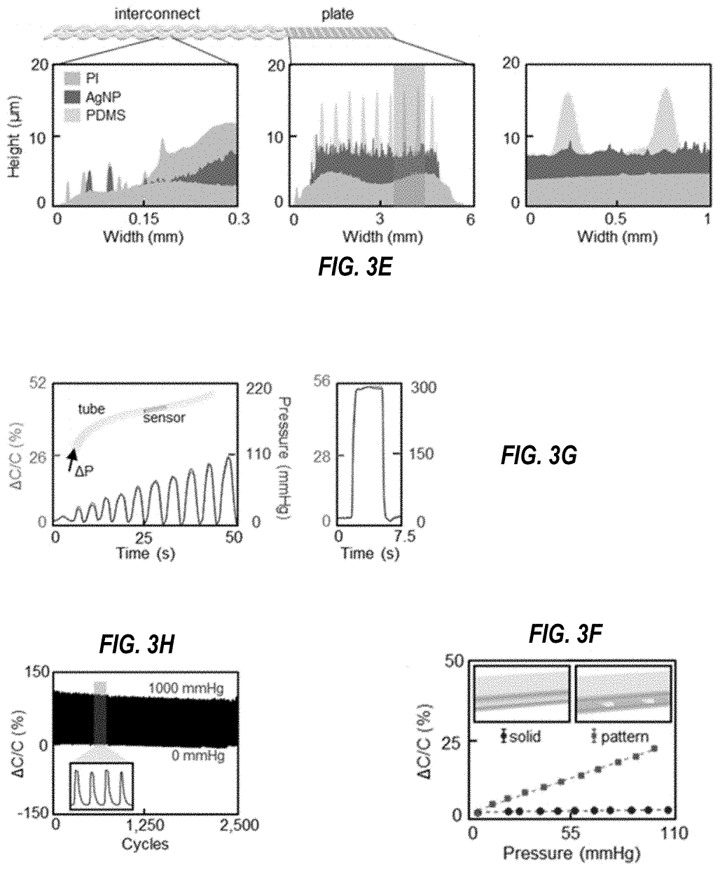

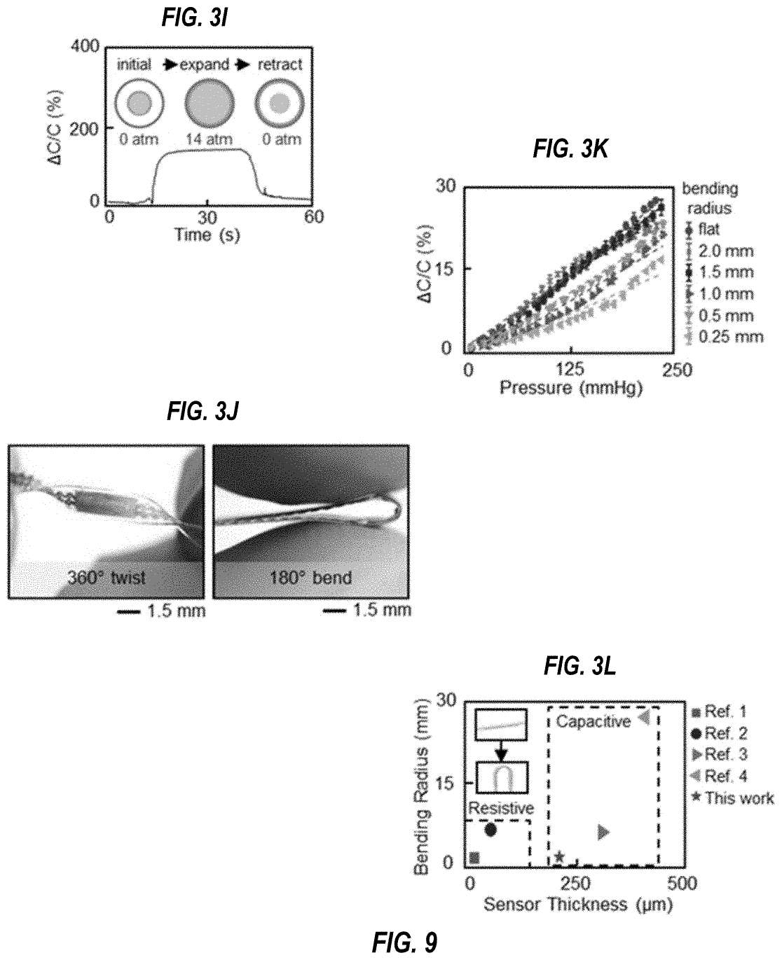

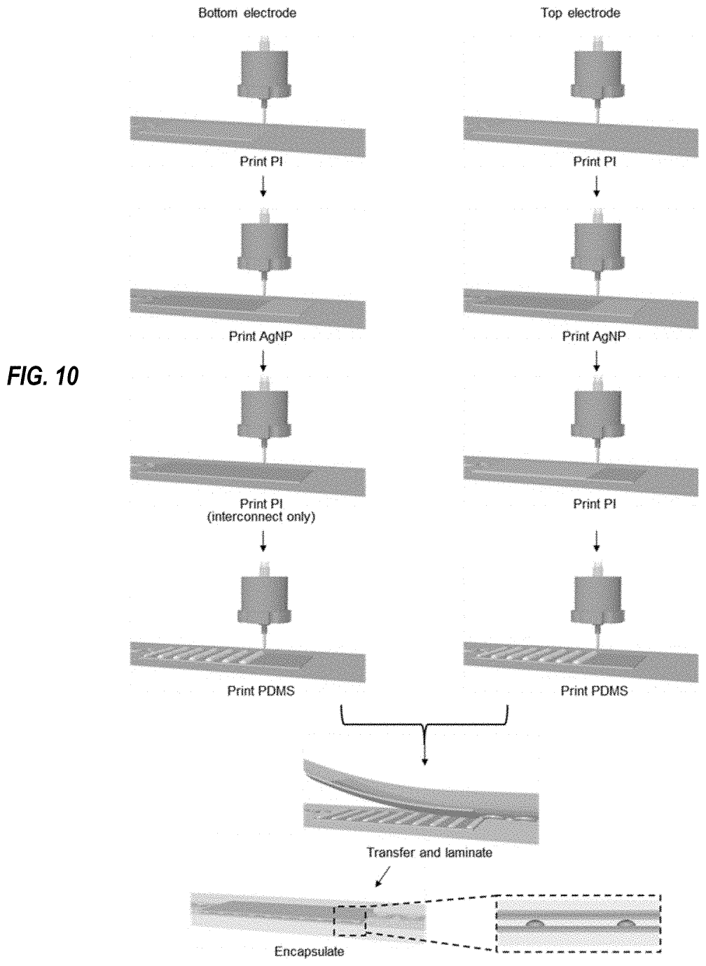

An aerosol jet printing method can enable a fully printed sensor by taking advantage of its rapid fabrication process compatible with a wide range of ink viscosities from 1 to 1000 cP. FIG. 3A illustrates the printed layers of PI, silver nanoparticles (AgNP), and polydimethylsiloxane (PDMS) forming the pressure sensor with stretchable interconnects (details in Materials and Methods below and also shown in FIG. 10). The top and bottom electrodes can be printed separately on the same substrate via a nozzle, as shown in FIG. 3B. Following printing, the two layers can be transferred and laminated together in elastomer, and the completed sensor is shown in FIG. 3C. PDMS encapsulation was used due to the known hemocompatibility, which may be enhanced with surface modifications, though other encapsulation materials can be employed. Ink and printing parameters were optimized to achieve a thin and durable sensor, as shown in Table 1. A structured dielectric layer was printed with PDMS on the bottom electrode, as shown by the SEM image in FIG. 3D. Following prior reports of molding PDMS microstructures to enhance pressure sensitivity, here printing replaces molding to achieve rapid fabrication with fewer processes and direct PDMS patterning. Interconnect thickness can be less than 12 μm, while the bottom electrode and dielectric can be less than 16 μm thick (FIG. 3E). The printed PDMS structures show uniformity and could be continuously printed on numerous sensors. Printing speed and the number of passes can be controlled to adjust the height and width of PDMS traces.

TABLE 1

| Parameter | PI | AgNP | PDMS |
|---|---|---|---|
| Ink Solvent | NMP | m-Xylene | Toluene |
| Sheath Rate (ccm) | 20 | 40 | 30 |
| Atomization Rate (ccm) | 1100 (exhaust) | 40 | 1100 (exhaust) |
| | 1150 (atomiza- | | 1115 (atomiza- |
| | tion) | | tion) |
| Nozzle Diameter | 300 | 200 | 200 |
| Printing Speed (mm s⁻¹) | 10 | 10 | 15 |
| Printing Passes | 5 | 6 | 9 |
| Stage Temperature | 80 | 65 | 80 |
| (° C.) | | | |
| Curing/Sintering | 240 | 240 | 100 |
| Temperature (° C.) | | | |

The dielectric layer of printed PDMS lines can offer significantly higher pressure sensitivity compared to a solid film, as shown in FIG. 3F, and can achieve an average sensitivity of 0.013 kPa⁻¹. The improvement stems from the PDMS structures having space to deform under pressure. Capacitance values are shown as capacitance change divided by baseline capacitance (ΔC/C₀). Typical baseline capacitances were 3-6 pF. The capacitive sensors indicated impacts of stray capacitance during wired tests, but these effects were not observed once wires were removed and sensors were integrated with the wireless stent. A variety of PDMS patterns were printed, but the manual assembly of thin electrode results indicated similar sensitivities. All sensor results shown use printed PDMS lines with 525 μm centerline spacing.

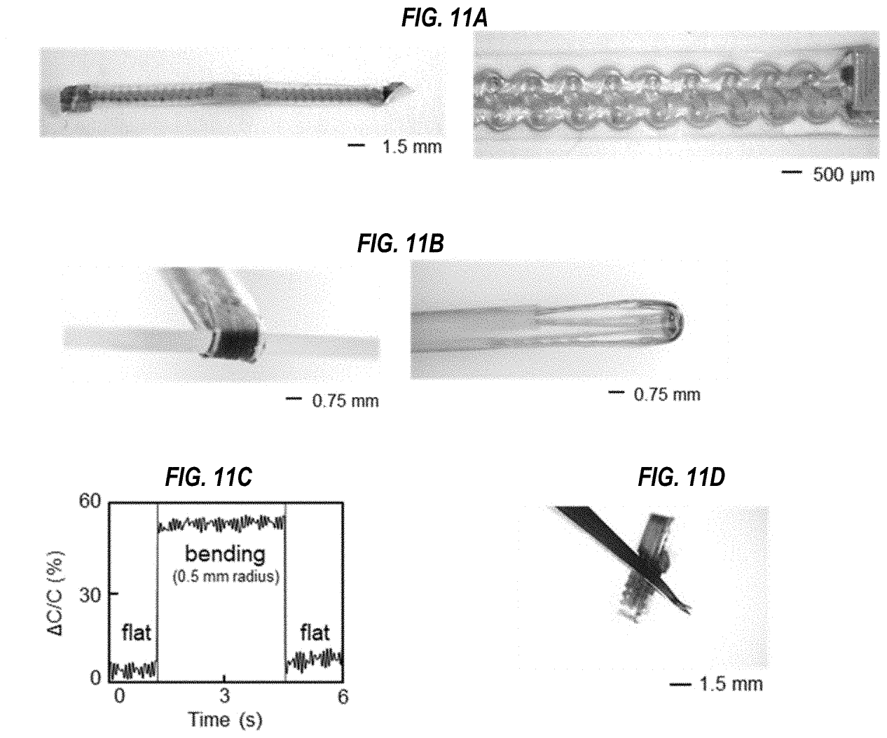

The sensor can detect continuous pressure changes and displays an immediate response time even at high pressures (FIG. 2G). A solid dielectric layer can slow response time and lower sensitivity. The sensors can display durability to cyclic pressure varied from 0 to 1,000 mmHg, which is over 5× larger than artery pressures (FIG. 3H). In addition, in some applications, it can be critical for the sensor to withstand the pressure exerted onto a stent during balloon expansion. FIG. 3I shows the sensor capacitance during stent deployment with a balloon pressure maintained at 14 atm for 30 seconds, which exceeds the typical time and pressure needed to expand the inductive stent. Sensor capacitance increases and quickly decreases with a 3.5% baseline change after 60 s. The thin printed layers embedded in the elastomer can offer a highly flexible sensor capable of twisting and bending without failure (as shown in FIG. 3J). The sensor can conform to bending radii smaller than 0.5 mm and recovers baseline capacitance, as shown in FIG. 11A-D. While prior pressure sensors offered flexible formats, many show sensitivity losses or are not demonstrated to sense pressure in a bending state. By using thin active sensor layers and disconnected PDMS microstructures as the dielectric layer, the pressure sensor can conform to a bending radius of 1.5 mm without loss of sensitivity, as shown in FIG. 3K. Pressure sensing is demonstrated to a 0.25 mm bending radius, which is 20× smaller than prior capacitive pressure sensors and the second smallest among resistive pressure sensors.

Demonstration of a Wireless Device in an Artery Model

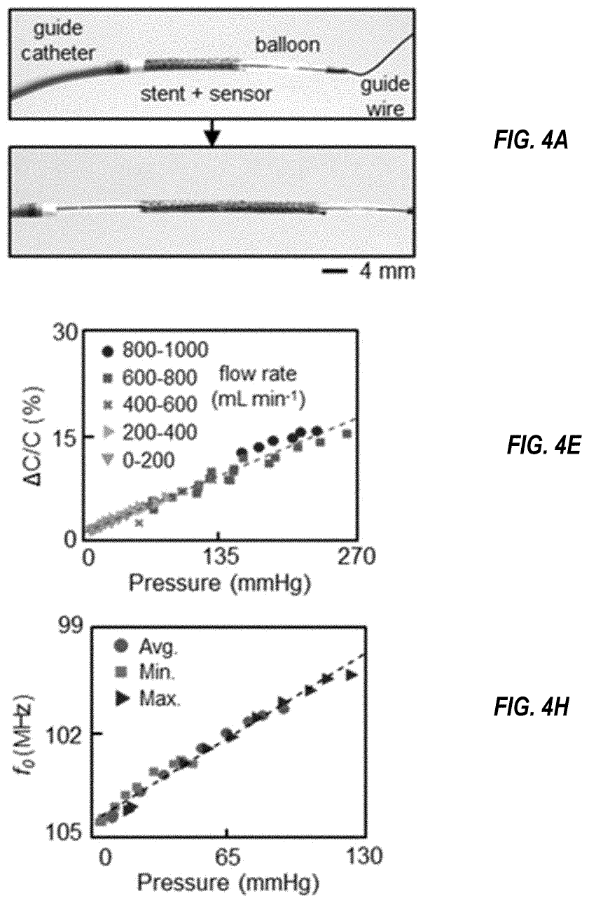

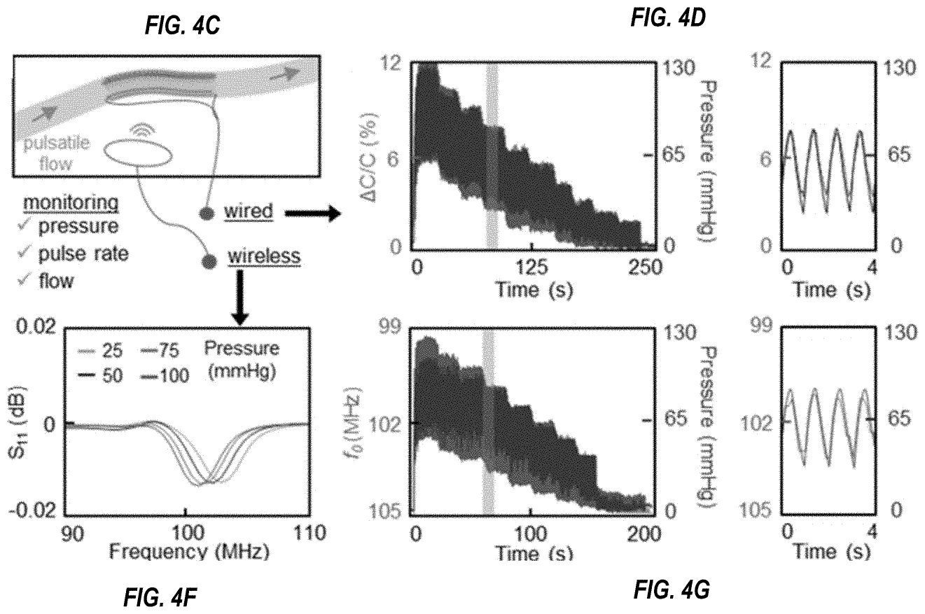

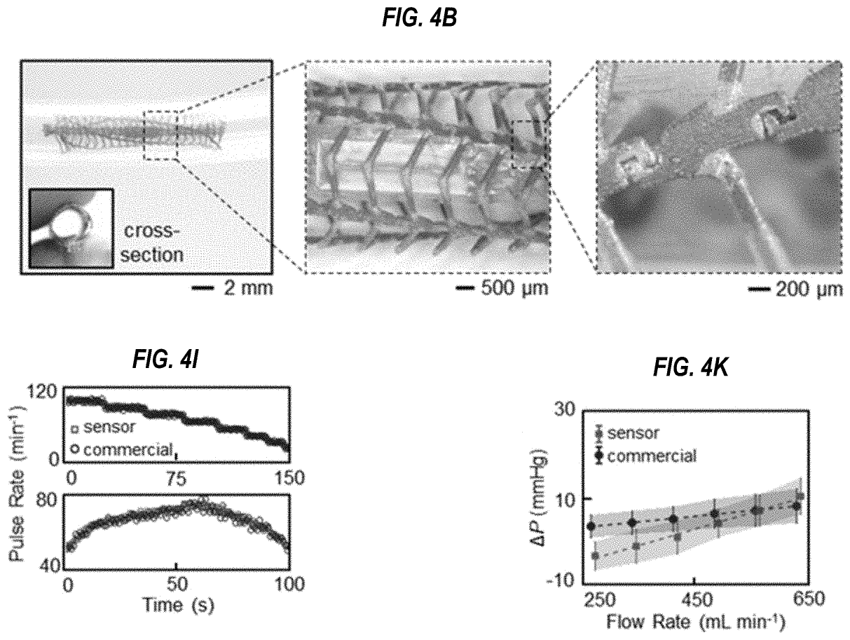

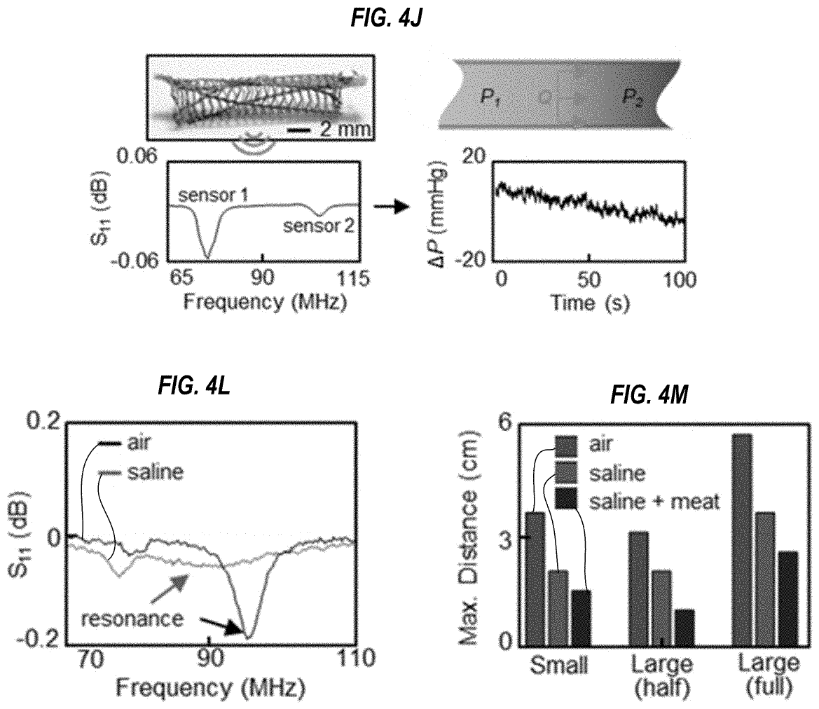

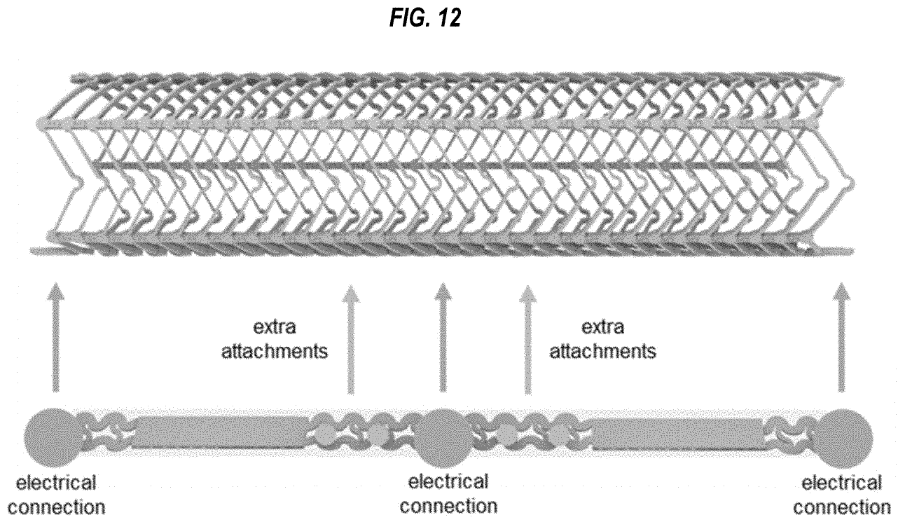

For implantation, the sensor can be integrated within the stent and connected at each end to complete the LC circuit before crimping onto a balloon catheter and advancing through a guide catheter (FIG. 4A). The narrow sensor can allow for attachment along the length of the stent to avoid significant deformation of the sensor during stent expansion, as shown in FIG. 12. The low-profile system can be expanded into a silicone artery connected to a pulsatile pump to create physiological conditions, as shown in FIG. 4B.

The stent and sensor were validated through wired monitoring of capacitance and wireless monitoring of resonant frequency, as illustrated in FIG. 4C. FIG. 4D shows wired monitoring of sensor capacitance closely following the pulsatile pressure. A variety of pressures, flow rates, and pulse rates were applied to the sensor. During sensing, the low-profile form of the sensor avoids flow noise interferences despite large flow rate changes from 0 to 1,000 mL min⁻¹, as shown in FIG. 4E. Wireless monitoring can be achieved by continuously measuring the Su parameter of the device with an external loop antenna and VNA. FIG. 4F shows frequency sweeps of a device in the artery model at different pressures during pulsatile flow. Baseline resonant frequencies of devices in the artery model ranged from 70-110 MHz depending on stent inductance and sensor capacitance, though the disclosure is not so limited. Continuous collection of frequency sweeps enables real-time monitoring of arterial pressure, as demonstrated in FIG. 4G. Owing to the fast response time of the pressure sensors, the device wirelessly detects the average, minimum, and maximum values of each pulsatile pressure waveform (FIG. 4H). Measurements in static air pressures demonstrated a wireless resolution as low as 5 mmHg, along with detection of sudden and large pressure changes. In the artery model, the wireless system monitored changes in system pressure, flow rate, and pulse rate, along with sudden, abnormal changes. With real-time pressure monitoring, pulse rates can be simultaneously monitored by evaluating the recorded pulsatile wave frequency. FIG. 4I compares pulse rates calculated using wireless device signals and using wired signals from a commercial pressure sensor.

Fitting the stent with two pressure sensors enables monitoring of flow rate changes in an artery. Each pressure sensor is located proximate the stent ends to detect a pressure gradient across the length of the stent. By electrically connecting the pressure sensors together at the center of the stent, the stent is split into two inductors and allows for monitoring two distinct resonant frequencies (e.g., 72 MHz and 105 MHz) in order to determine a pressure gradient, as shown in FIG. 4J. FIG. 4K shows different flow rates, the average pressure gradient, and amplitude measured by the exemplary wireless device, which is similar to commercial pressure sensors. The difference in linearity may arise from the delay between wirelessly measuring the two sensors, wireless resolution, minor pressure changes within the stent, and the obtrusive attachment of commercial sensors to the artery. The increased difference at lower flow rates is expected to be caused by the increasingly smaller pressure gradient and can be remedied by precise calibration at lower pressures.

The wireless system provided similar pressure values and captured flow changes, which indicates the ability to estimate flow rate and physiological changes, such as restenosis. Wireless performance when operated in blood and tissue was characterized. When operated in blood, the conductivity of blood dampens the inductive stent signal. While a thick parylene coating decreases this effect, operation in a saline concentration matching the conductivity of blood dampens the wireless signal, as shown in FIG. 4L. As a result, the readout distances are approximately halved when operating in a saline and through tissue surroundings (FIG. 4M). The distance of 5.5 cm in air and 3.5 cm in saline is an improvement over existing stent-based devices, which have been limited up to 3 cm in air and 1 cm in the blood. Compared to prior works, the presented device offers more comprehensive sensing of normal artery hemodynamics at the best readout distances while maintaining a low-profile and demonstrates a thorough in vivo catheter implantation for stent-based devices.

In Vivo Study of Device Implantation Via a Catheter

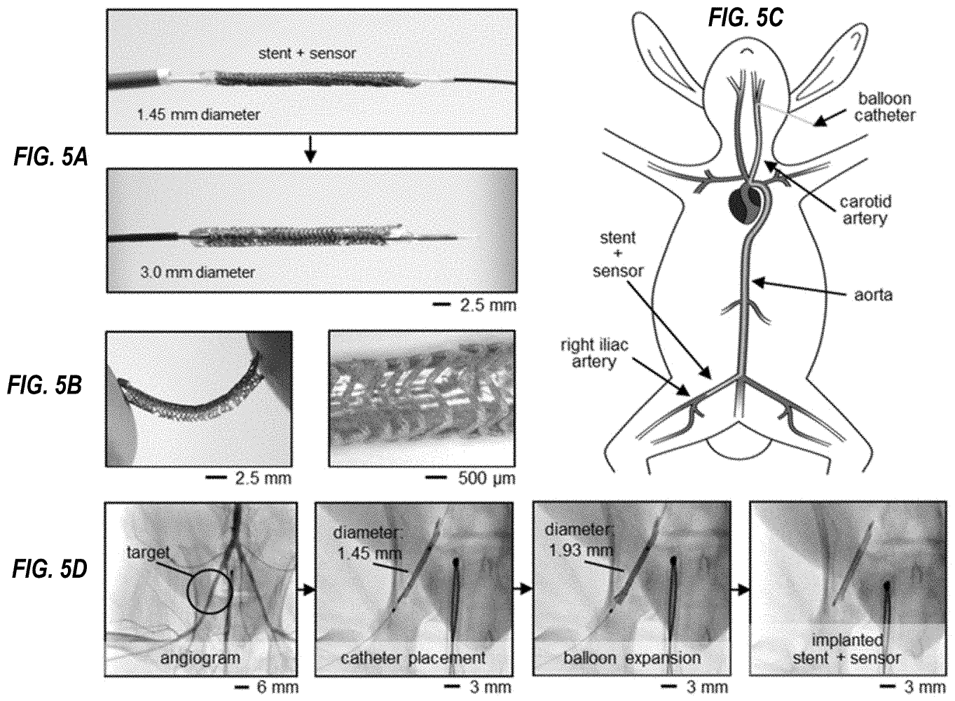

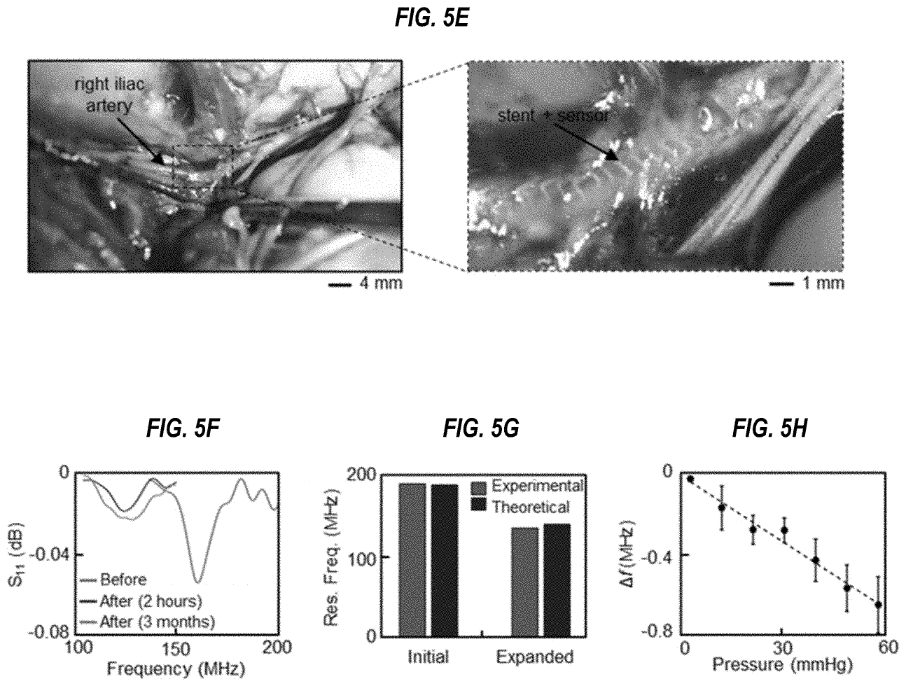

An in vivo rabbit study was performed to demonstrate catheter deployment. For implantation, a small inductive stent with an initial diameter below 1.5 mm and an expanded diameter up to 3.0 mm was used (FIG. 5A). An expanded device is shown in FIG. 5B with an enlarged view of the stent and a further miniaturized printed pressure sensor. In prior works on vascular sensors delivered by catheter, in vivo studies have been limited to minimal catheter advancement, failed implantations, and the use of surgical grafts. Here, the wireless device was guided from a vascular sheath insertion site in the left carotid artery, over the aortic arch, through the abdominal aorta, and into the right iliac artery (FIG. 5C). This route is the most extensive implant demonstration via catheter for existing stent-based devices. The pathway highlights the ability of the wireless device to advance through narrow and curved arteries with an aorta diameter of 4.5 mm and a right iliac artery diameter of 1.93 mm. FIG. 5D shows fluoroscopy images during expansion and after catheter removal in the right iliac artery. FIG. 5E shows photos of the implanted device in the right iliac artery following the in vivo study. Additionally, a second wireless device was able to be guided around a sharp turn and into the left renal artery with a diameter of 1.53 mm, indicating the potential for sensing in highly narrow arteries. Following the in vivo study, the right iliac artery was harvested to confirm the functionality of the implanted sensor. FIG. 5F shows wireless signals from the stent before implantation, 2 hours after removal, and 3 months after removal. The resonance signals in FIG. 5F differ from the prior signals shown in FIG. 4F due to stent size differences. Signals shown in FIG. 4F were collected with a stent expanded to a 5 mm diameter, while signals in FIG. 5F were collected with a stent expanded to a 2 mm diameter. The smaller stent diameter causes a decrease in inductance and an increase in resonant frequency. Additionally, variations in sensor base capacitance shift the frequency range of devices. A laminating press can be used to more uniformly seal sensors and will improve the consistency of the devices. The shift in resonant frequency from 175 MHz to 135 MHz due to expansion compares well with theoretical calculations (FIG. 5G). For the implanted device, sensor capacitance and initial stent inductance were 5.3 pF and 0.15 μH, respectively. Based on these parameters, the theoretical resonant frequency of the device before expansion is 178 MHz. Following expansion from a diameter of 1.5 mm to 2 mm, the stent inductance is expected to increase to 0.26 μH, which shifts the resonant frequency to 136 MHz. Similar to that observed in vivo, pressure changes were applied to the harvested device and artery to ensure pressure sensing functionality. FIG. 5H indicates the implanted device's change in resonant frequency with low-pressure ranges.

As discussed above, disclosed herein are fully implantable, vascular electronic systems comprising a wireless stent platform and printed soft sensors for real-time sensing of arterial pressure, pulse rate, and flow. Design, materials, and fabrication strategies of the inductive stent are developed to enhance wireless capabilities while maintaining key aspects of conventional stents. The fully printed capacitive sensors with microstructured features enable a significant improvement in pressure sensing during bending due to the thin, flexible layers and patterned PDMS. The wireless device demonstrates multiplex sensing of hemodynamics at extended readout distances in an artery model. An in vivo rabbit study shows minimally invasive catheter implantation in narrow arteries. Though the wireless implantable device platform is disclosed herein as used to monitor hemodynamic properties, the disclosure is not so limited. The devices can also be readily adaptable for a multitude of sensors to monitor more parameters, such as strain, temperature, and biomarkers, and would allow for disease-specific devices.

### Examples

Below we describe certain exemplary devices and methods of fabrication. These examples are exemplary only and should not be construed as limited the scope of the present disclosure.

Materials and Methods

Fabrication of inductive stent. The inductive stent was fabricated with a femtosecond laser (Optec) using a tubing cutting stage. Stainless steel tubing (Vita Needle) with an outer diameter of 2.1 mm and wall thickness of 76 μm was the first laser machined using a 60% power, a speed of 3.6 mm s⁻¹, and 5 passes to form holes for the connectors. Following cutting, the tubing was sonicated in DI water to remove debris and clean the machined surfaces. Electropolishing was performed for 45 s with a current of 0.6 A in the electropolishing solution (E972, ESMA). The polished tubing was then rinsed with DI water and dried. The tubing was then dip-coated in polyimide (PI; HD MicroSystems, PI-2545) prior to curing at 240° C. for 1 hour. Dip coating and curing were then completed a second time to ensure full coverage. Following PI coating, sanding the surfaces of the tubing removed excess PI. The tubing was then laser machined at identical parameters to form the final stent structure. Sonication in DI water and electropolishing of the stent structure was performed with identical parameters to clean surfaces. Surface plating of a 20 μm thick layer of Au was performed by electrodeposition using a three-electrode system with a reference electrode (commercial Ag/AgCl electrode), Pt counter electrode, and the electropolished stent as a working electrode. The electrodes were submerged into a bright electroless gold plating solution (Sigma Aldrich), and cyclic voltammetry deposition was conducted via a potentiostat (Gamry 1010E). During the deposition, the temperature and pH of the plating solution were controlled at 55° C. and 8, respectively. The potential was swept from −0.65 to −0.95 V versus the commercial Ag/AgCl electrode for 850 cycles at a scan rate of 0.05 V s⁻¹. The surface of the Au-deposited stents was thoroughly rinsed by DI water to remove chemical residues that are potentially active and harmful in the implant circumstance. Following electroplating, a 25 μm thick layer of parylene was deposited onto the stent using a parylene coater (SCS Labcoter).

Stent characterization. Balloon expansion was performed with a 5 mm diameter balloon catheter (Cook Advance 18LP PTA) and an inflator with a pressure gauge filled with DI water. Small stents with an initial diameter of 1.5 mm used a 2 mm diameter balloon catheter (Cordis Savvy Long PTA). Inductance was measured using an LCR meter (B&K Precision 891), and resistance was measured using a multimeter (Keithley DMM7510). Wireless frequency sweeps of the S₁₁ parameter were recorded with a vector network analyzer (VNA; Tektronix TTR506A) controlled by a custom Matlab program in order to determine the resonant frequency. The resonant frequency was determined by locating the minimum of the S₁₁ parameter after subtracting a baseline frequency sweep. Loop reader antennas were formed with a single loop of Cu wire and connected to the VNA for recording. For performance comparison between a stent and Cu coil, the Cu coil was created by wrapping Cu wire around plastic tubing with a diameter, number of turns, and length equal to the stent. Noise levels were measured at frequencies lower and higher than resonance. Readout distances were measured for inductive stents connected to printed pressure sensors. Axial readout distance was measured by recording frequency sweeps while increasing the axial distance between the stent and external reader antenna. Radial readout distance was measured by recording frequency sweeps with different reader antenna diameters and placing the stent at the center of the reader antenna. To improve readout distance, external reader antennas were tuned with discrete ceramic capacitors to the resonant frequency of the stent and sensor. Stent's mechanical stiffness was measured with a motorized vertical test stand (Mark-10 ESM303) and force gauge (Marl-10 M5-5). The stage was moved by a set displacement while recording force. All stent samples, including the commercial stent (Medtronic Visi-Pro), were expanded to 4.5 mm in diameter.

Fabrication of soft pressure sensors. An aerosol jet printing system (Optomec 200) was used to print sensor layers. First, a layer of polymethyl-methacrylate (PMMA; MicroChem) was spin-coated on a glass slide at 3,000 r.p.m. for 30 s and cured at 180° C. for 3 minutes. The support layer of PI was printed via the pneumatic atomizer with parameters in Table S1. The PI ink was formed in a 3.5:1 mixture of PI to 1-methyl-2-pyrrolidinone (NMP; Sigma Aldrich). The bottom layer of PI was then cured in an oven at 240° C. for 1 hour. Following curing, the printed PI was plasma treated for 1 minute before printing AgNP ink (UTDOTS, AgNP40X) via the ultrasonic atomizer with parameters in Table 1 (above). The AgNP layer was sintered at 240° C. for 1 hour. After sintering, a top layer of PI was printed and cured with identical parameters. Printing of polydimethylsiloxane (PDMS; Sylgard 184, Dow Corning) with the pneumatic atomizer and parameters in Table 1 was then performed on the bottom electrode area of the sensor. PDMS ink was formed with an 18:4 mixture of 10:1 (base to cure) PDMS and toluene (StarTex). Printed PDMS was cured at 100° C. for 1 hour. Following printing, the glass slide was covered and placed in an acetone bath for at least 1 hour to dissolve the underlying PMMA layer. After removing from the acetone bath, the sensors were transferred and aligned with tweezers onto elastomer. For transferring, the bottom electrode was first placed onto the elastomer with the PDMS dielectric layer facing up. The top electrode was then aligned and stacked on top of the bottom electrode. A small amount of PDMS was applied and cured along the interconnects to keep the sensor layers in place on the elastomer substrate. To seal the sensors, a piece of elastomer substrate was cut and laminated over the electrode area. A small amount of PDMS was poured and cured along the edges and interconnects while applying pressure to the elastomer piece covering the electrodes. After curing, the assembled and sealed sensor was removed from the plastic dish. Cu wires were attached to the interconnects with silver paint for wired sensing. The sensor was attached inside the stent for wireless sensing and connected to each end of the stent and the center of the stent with silver paint. A small amount of PDMS was used to insulate the electrical connections and to provide additional attachment points along the length of the sensor.

Sensor characterization. Sensor capacitance was recorded with the LCR meter. Pressure response was characterized by placing the sensors in silicone tubing connected with a syringe. The pressure was applied by displacing the syringe while a commercial sensor (Honeywell 26PCBFB6G) recorded pressure. Pressure sensing during a bending state was accomplished by bending the sensor around glass slides and taping the sensor at both ends away from the bending area. Glass slides with a thickness of 1.0 mm were stacked and used for bending radii between 0.5 mm and 2.0 mm. A bending radius of 0.25 mm was maintained by taping the sensor interconnects together without a spacer in between. The pressure was then applied by displacing the syringe. Cyclic tests were performed using the motorized vertical test stand attached with a force gauge. The vertical stage applied pressure onto a sensor while the LCR meter recorded capacitance. Compatibility with balloon catheter expansion was validated by attaching the sensor inside a stent. The stent was then expanded against the wall of silicone tubing while recording capacitance.

Wireless sensing in artery model. An artery model, with a wireless device expanded within, was formed with silicone tubing connected to a pulsatile pump (Harvard Apparatus). Valves were included upstream and downstream of the wireless device to modify system pressure while the pump was used to modify pulse rate from 0 to 120 min⁻¹ and stroke volume from 0 to 10 mL. The flow of both DI water and saline were used to characterize sensing. A commercial pressure sensor was located near the sensor and stent to record pressure simultaneously. Wired measurements used an LCR meter while wireless measurements used a VNA. The antenna was placed around the silicone artery and aligned with the stent for wireless sensing. Pulse rate was calculated by determining the maximum and minimum values of the recorded pressure and capacitance waveform. The time difference between the two was determined and converted to a pulse rate. A pressure gradient was wireless measured by recording the resonant frequency of each pressure sensor simultaneously. Prior to testing in flow, the resonant frequency of each sensor was measured for static pressure. By using static pressures, a calibration curve of resonant frequency and pressure was created for each sensor. During wireless recording in flow, the resonant frequency of each sensor was converted to pressure by using its calibration curve. The pressure difference between the sensors was then determined at each time point by subtracting the two pressure values. The calculated pressure difference determined the average pressure gradient and amplitude of the pressure gradient. For comparison, two commercial pressure sensors were located at a distance equal to the wireless device's sensors. The pressure gradient between the two commercial sensors was recorded. The wireless device was characterized when implanted in saline and meat to replicate in vivo conditions of blood and tissue. A saline concentration of 0.08 M was used to match the conductivity to blood. The meat was wrapped around the artery model to the specified thickness and extended more than 4 cm away from the implanted stent in both directions along the axial length.

In vivo demonstration. A New Zealand white rabbit was used in accordance with the approved protocol (#GT69B, T3 Labs, Global Center for Medical Innovation). Under inhalant isoflurane anesthesia, a vascular sheath was placed in the left carotid artery. The animal was then heparinized to achieve an active clotting time over 250 s. The device was mounted on a balloon catheter (Cordis Savvy Long PTA) and advanced over a 0.018 in. guidewire with fluoroscopic visualization. The device was advanced from the left carotid artery, over the aortic arch, and through the abdominal aorta to reach the targeted right iliac artery. The device was expanded with a balloon catheter pressure of 10 atm before removal of the catheter. The animal was monitored during the study. In vivo wireless measurements were found to be unreliable due to the small artery size and distance between the implanted device and skin. Following the in vivo study, the right iliac artery was harvested and stored in 10% neutral buffered formalin. The harvested device was maintained in the right iliac artery and placed inside silicone tubing for wireless testing of pressure sensing. Wireless signals were recorded 2 hours after harvesting and 3 months after harvesting. Wireless signal noise was removed using a low pass filter and a cubic smoothing spline. The pressure was applied by displacing a syringe while a commercial pressure sensor was simultaneously recorded. Wireless signals were collected with a loop antenna and VNA.

It is to be understood that the embodiments and claims disclosed herein are not limited in their application to the details of construction and arrangement of the components set forth in the description and illustrated in the drawings. Rather, the description and the drawings provide examples of the embodiments envisioned. The embodiments and claims disclosed herein are further capable of other embodiments and of being practiced and carried out in various ways. Also, it is to be understood that the phraseology and terminology employed herein are for the purposes of description and should not be regarded as limiting the claims.

Accordingly, those skilled in the art will appreciate that the conception upon which the application and claims are based may be readily utilized as a basis for the design of other structures, methods, and systems for carrying out the several purposes of the embodiments and claims presented in this application. It is important, therefore, that the claims be regarded as including such equivalent constructions.

Furthermore, the purpose of the foregoing Abstract is to enable the United States Patent and Trademark Office and the public generally, and especially including the practitioners in the art who are not familiar with patent and legal terms or phraseology, to determine quickly from a cursory inspection the nature and essence of the technical disclosure of the application. The Abstract is neither intended to define the claims of the application, nor is it intended to be limiting to the scope of the claims in any way.

What is claimed is:

1. A wireless hemodynamic sensor system, comprising:
a stent having an outer perimeter defining an interior volume;
a sensor member positioned along the outer perimeter, the sensor member comprising:
a first sensor positioned proximate a first end of the stent; and
a second sensor positioned proximate a second end of the stent,
wherein the sensor system is configured to simultaneously measure blood pressure, pulse rate, and blood flow rate of blood passing through the interior volume.

2. The sensor system of claim 1, wherein the outer perimeter of the stent comprises a plurality of conductive loops, each of the plurality of conductive loops coupled to an adjacent conductive loop via a non-conductive connector.

3. The sensor system of claim 2, wherein the outer perimeter forms an inductive antenna.

4. The sensor system of claim 1, wherein the sensor member comprises a first electrode, a second electrode, and a dielectric layer positioned between the first and second electrodes.

5. The sensor system of claim 4, wherein the sensor member is electrically coupled to the stent via a first connection to the first electrode proximate the first end of the stent, a second connection to the first electrode proximate the second end of the stent, and a third connection to the second electrode proximate a location between the first and second sensors.

6. The sensor system of claim 4, wherein each of the first, second, and third connections are insulated with PDMS.

7. The sensor system of claim 1, wherein the first sensor is configured to operate within a first resonant frequency range, and wherein the second sensor is configured to operate within a second resonant frequency range, wherein the first resonant frequency range does not overlap with the second resonant frequency range.

8. The sensor system of claim 1, wherein the system is configured to measure a pressure gradient between the first sensor and the second sensor.

9. The sensor system of claim 1, wherein the blood pressure, pulse rate, and blood flow rate measurements are not degraded if the sensor member are bent with a radius of curvature of 1.5 mm.

10. The sensor system of claim 1, wherein the first and second sensors are capacitive pressure sensors.

11. The sensor system of claim 1, wherein the plurality of conductive loops comprise stainless steel.

12. The sensor system of claim 11, wherein the plurality of conductive loops are coated in gold.

13. The sensor system of claim 1, wherein the nonconductive connectors comprise polyimide.

14. The sensor system of claim 1, wherein each of the plurality of conductive loops has an S-shape to facilitate stretching of the stent.

15. A wireless hemodynamic sensor system, comprising
a stent having an outer wall defining an interior volume, the stent configured to be placed in a blood vessel of a patient;
a sensor member positioned along inner surface of the outer wall, the sensor member comprising a first capacitive pressure sensor positioned proximate a first end of the stent and a second capacitive pressure sensor positioned proximate a second end of the stent,
wherein the first and second sensors are configured to measure blood pressure, blood flow rate, and pulse rate of blood flowing through the blood vessel.

16. The sensor system of claim 15, wherein the outer wall of the stent comprises a plurality of conductive loops, each of the conductive loops coupled to an adjacent conductive loop via a nonconductive connector, the outer wall forming an inductive antenna capable of being interrogated by a second external inductive antenna.

17. The sensor system of claim 15, wherein each of the plurality of conductive loops has an S-shape to facilitate stretching of the stent.

18. The sensor system of claim 15, wherein the sensor member comprises:
a first electrode electrically coupled to the first and second ends of the stent;
a second electrode electrically coupled the stent between the first and second ends of the stent; and
a dielectric material between the first and second electrodes.

19. The sensor system of claim 15, wherein the sensor system is capable of measuring blood pressure, blood flow rate, and pulse rate of blood flowing through the blood vessel if the sensor member is bent at a radius of 1.5 mm.

20. The sensor system of claim 15, wherein the first sensor is configured to operate within a first resonant frequency range, and wherein the second sensor is configured to operate within a second resonant frequency range, wherein the first resonant frequency range does not overlap with the second resonant frequency range.
